SEQUENCE LISTING (9 sequences)

SEQ ID NO: 1 - DNA, 22 bp, Artificial Sequence
  Description of Artificial Sequence Synthetic primer
ctgatggatgaagtgcaattac

SEQ ID NO: 2 - DNA, 19 bp, Artificial Sequence
  Description of Artificial Sequence Synthetic primer
cagcctctgcaactgtctg

SEQ ID NO: 3 - DNA, 18 bp, Artificial Sequence
  Description of Artificial Sequence Synthetic primer
gatctacgggccaatctc

SEQ ID NO: 4 - DNA, 20 bp, Artificial Sequence
  Description of Artificial Sequence Synthetic primer
ggctgtggtacatcagtact

SEQ ID NO: 5 - DNA, 19 bp, Artificial Sequence
  Description of Artificial Sequence Synthetic primer
cctctggaggcaggagaag

SEQ ID NO: 6 - DNA, 21 bp, Artificial Sequence
  Description of Artificial Sequence Synthetic primer
gctctggatataaccctcaga

SEQ ID NO: 7 - DNA, 21 bp, Artificial Sequence
  Description of Artificial Sequence Synthetic primer
tccgtagatctctgttgatac

SEQ ID NO: 8 - DNA, 19 bp, Artificial Sequence
  Description of Artificial Sequence Synthetic primer
cacactgctgcagagcatg

SEQ ID NO: 9 - DNA, 21 bp, Artificial Sequence
  Description of Artificial Sequence Synthetic primer
tgatactaagaccagcatgct

(19) United States
(12) Patent Application Publication
(10) Pub. No.: US 2007/0087343 A1
(43) Pub. Date: Apr. 19, 2007

(54) Method of detecting gene expression and kit and apparatus to be used therein

(75) Inventors: Motoyuki Ogawa, Tokyo (JP); Yoshiki Hiraoka, Tokyo (JP); Masanori Hasegawa, Kanagawa (JP); Sadakazu Aiso, Tokyo (JP)

Correspondence Address: BARNES & THORNBURG LLP, 11 SOUTH MERIDIAN, INDIANAPOLIS, IN, 46204, US

(73) Assignee: KEIO UNIVERSITY, Tokyo (JP)

(21) Appl. No.: 10/513,809

(22) PCT Filed: May 9, 2003
(86) PCT No.: PCT/JP03/05835

§ 371 Date: Oct. 4, 2005

(30) Foreign Application Priority Data
May 9, 2002 (JP) 2002-134388

(51) Int. Cl. C12Q 1/68; A01K 67/027

(52) U.S. Cl. 435/6; 800/14; 800/18

(57) ABSTRACT

In an animal embryo or tissue, the following wholeamount treatment is carried out so that gene expression is detected. First, a sample such as an embryo or a tissue is fixed and then pretreated by acetylating an amine with acetic anhydride and eliminating lipids by using xylene. Next, in situ RT-PCR is performed with the use of a dNTP/analog mixture in an mRNA Selective PCR Kit™ (manufactured by TAKARA) and dig-dUTP™ (manufactured by Roche), under modification conditions at 85° C. As a result, genomic DNA is not amplified but DNA originating in mRNA—is specifically amplified and labeled. Finally, the thus amplified DNA is detected by using digoxigenin as an indication. Thus, it is possible to provide a method of detecting gene expression whereby three-dimensional results can be obtained in an animal embryo or tissue at a high detection sensitivity by simple procedures and a kit and an apparatus to be used therein.

### TECHNICAL FIELD

The present invention relates to methods for detecting gene expression in animal embryos or tissues, and kits and apparatus for use therefor.

### DEFINITIONS

“Gene” means the region that includes one transcription unit and the regulatory regions of its expression on genomic DNA, consisting mainly of a transcriptional region, which includes exons containing coding regions that code for proteins as well as non-coding regions flanking them and intervening introns, together with enhancer/promoter region(s) controlling expression of the transcriptional region. mRNA is formed as a result of removal of the intron portions from RNA transcripts and linking of the exon portions.

“cDNA” means DNA that has the complementary sequence to the whole or a part of mRNA. It may contain not only DNA synthesized by reverse transcription of the mRNA but also a complementary polynucleotide synthesized using a complementary polynucleotide as a template, which has been synthesized using the mRNA as a template.

“Whole-mount” means not making thin-sliced sections (typically a few micrometers to a few dozens of micrometers in thickness) using a microtome or the like. A whole-mount embryo or tissue refers not only to an embryo as a whole taken out but also to its head only, idiosoma from which the head has been cut off, the whole tissue taken out from an individual, a divided tissue block, or the like. In addition, a whole-mount embryo or tissue may consist of a single or multiple tissues and it (they) may compose an organ. Even if it is a tissue cut with a razor etc., in protocols for thin sections, such a cut tissue section thick enough to the extent that a probe or the like does not permeate inside is included in whole-mount tissues as defined herein.

“PCR” generally refers to a methods such as that described in U.S. Pat. No. 4,683,195, i.e., the method for enzymatically amplifying the target nucleotide sequence, consisting of multiple, typically 10-40 cycles of the denaturation step in which the DNA duplex dissociates, the annealing step in which primers hybridize to single-stranded DNA by hydrogen bonds, and the extension step in which polymerases synthesize DNA from primers by using single-stranded DNA as a template.

### BACKGROUND ART

While the genome projects of various animals are under way, a multitude of genes with unknown functions are being identified using genome information or cDNA information. The conventional methodology has also served to, in many cases, isolate genes (e.g. oncogenes) or cDNA before their functions in the normal living body have been elucidated. When functional analysis is performed of such genes whose functions are less understood in the living body, the expression pattern of the genes is a big key.

One method for investigating expression of a certain gene in animal embryos or tissues is the in situ hybridization.

This method uses a DNA probe directly labeled with cDNA of the target gene or an RNA probe synthesized in vitro using the cDNA and labeled. The label contained in the probe is detected by hybridizing such a DNA/RNA probe with mRNA that has been transcribed on sections of the embryos or tissues. However, the problem with this method is that when the number of samples increases the complicated work of preparing hundreds of thin sections is required. Furthermore, analysis results obtained are the information on sections; in order to find how the target gene is three-dimensionally expressed in embryos or tissues, it is necessary to conduct experiments with serial sections, subsequently to create 3D stereoscopic configuration, and to reconstruct the results on slices in three dimensions.

Thus, the technique of in situ hybridization using whole-mount embryos or tissues has been developed. However, the technique presented a large number of technical problems such as poor permeation of probes or reagents and too many nonspecific bindings of nucleic acid due to thickness of whole-mount samples. To overcome these problems, technological development of whole-mount embryos or tissues took as long as more than five years after the development of the technique with slices in spite of world-wide research activities.

Still, whether the subject is a section or a whole-mount embryo or tissue, since in situ hybridization directly detects mRNA, there is a limit on detection sensitivity; especially, detection of mRNA with a low expression level is very problematic.

To solve this problem, a method of reverse transcribing mRNA and detecting nucleic acid obtained by amplifying resulting cDNA on embryo or tissue sections has been developed. This methods is called in situ PCR, RT in situ PCR, or in some cases, in situ RT-PCR (hereinafter called in situ RT-PCR), in which because of detecting, by amplification, mRNA which is transcripts, detection sensitivity for expression has greatly improved as compared with in situ hybridization.

Nevertheless, the method encompasses only the technique performed on thin sections and has the same problems as the above-mentioned in situ hybridization: necessity of preparing thin sections and creating 3D configuration, complicated operations, leading to low efficiency to obtain results. Moreover, although the method is performed on sections, its technical difficulties have hindered establishment of optimal conditions and very few reliable protocols are available. In situ RT-PCR is an underdeveloped technique.

Thus, the object of the present invention is to provide a method for detection of gene expression that has a high detection sensitivity, is easy to operate, and provides 3D results, together with kits and apparatus to be used for such a method.

### DISCLOSURE OF INVENTION

The method for detecting gene expression according to the present invention is a method for detecting gene expression including the steps of: in an animal embryo or tissue, synthesizing a cDNA from an RNA, amplifying a nucleic acid using the cDNA synthesized by the step of synthesizing the cDNA as a template, and detecting the nucleic acid amplified by the step of amplifying the nucleic acid, all of the steps being performed in a whole-mount animal embryo or tissue.

The animal may be a vertebrate.

The vertebrate may be Xenopus laevis or a mouse.

The step of amplifying the nucleic acid may include a denaturation step in which a nucleic acid duplex is denatured to a single strand, an annealing step in which a primer anneals to the nucleic acid that has became a single strand in the denaturation step, and an extension step in which a nucleic acid having a sequence complementary to the nucleic acid that has become a single strand is synthesized by using the nucleic acid that has become a single strand as a template and extending from the primer.

The step of amplifying the nucleic acid may be performed by polymerase chain reaction (PCR).

The whole-mount embryo or tissue may be vibrated while the nucleic acid is being amplified.

The nucleic acid may be amplified by specifically using the synthesized cDNA as a template in the extension step, without substantially denaturing genomic DNA in the denaturation step in the step of amplifying the nucleic acid.

The denaturation step may be performed at 90° C. or lower in the step of amplifying the nucleic acid.

The inclusion of dNTP analogue in a reaction system in the step of amplifying the nucleic acid may cause the dissociation temperature of the duplex consisting of the cDNA and its complementary strand generated in the extension step to be 90° C. or lower.

The dNTP analog may be derived from a dNTP/analog mixture in mRNA Selective PCR Kit (a trademark of TAKARA).

Another step of treating the whole-mount embryo or tissue with a protease may be included.

Another step of acetylating an amine in the whole-mount embryo or tissue may be included.

The step of acetylating the amine may include treating the whole-mount embryo or tissue with acetic anhydride.

Yet another step of removing a lipid in the whole-mount embryo or tissue may be included.

The step of removing the lipid may include treating the whole-mount embryo or tissue with xylene.

The method for detecting gene expression may be performed in a microcentrifuge tube or by using a multiwell plate.

In the method according to the present invention, the kit for detecting gene expression in a vertebrate embryo or tissue may be used, which includes: (a) a reverse transcriptase that synthesizes a cDNA from an RNA; (b) a nucleic acid polymerase that polymerizes a nucleic acid; (c) a dNTP analog that lowers the dissociation temperature of the nucleic acid than the dissociation temperature at which dNTP is used, when the dNTP analog is present in a double-stranded nucleic acid; and (d) a labeled dNTP.

The kit may contain (a) an antibody that recognizes the label portion in the labeled dNTP and that is conjugated to an enzyme for detecting a localization of the label; and (b) a substrate for the enzyme for detecting the localization.

A fixed embryo or tissue may be further included in the kit.

A dNTP/analog mixture in mRNA Selective PCR Kit may be included as the dNTP analog in the kit.

The labeled dNTP in the kit may be a dNTP labeled by digoxigenin.

The apparatus according to the present invention may be an apparatus for performing the above-described methods for detecting gene expression.

The apparatus may include a device for vibrating the whole-mount embryo or tissue while the nucleic acid is being amplified.

### BRIEF DESCRIPTION OF THE DRAWINGS

FIG. 1 shows a result of detection of the troponin I gene performed in accordance with one example of the present invention. Of the two embryos, the upper is a detected example and the lower is a negative control, to which no reverse transcriptase was added in reverse transcription reactions. In the upper embryo, heart-specific expression of the troponin I gene (the blue signal indicated by an arrow) has been detected.

FIG. 2 shows a result of detection of the Xlens1 gene performed in accordance with one example of the present invention. Of the two embryos, the upper is a detected example and the lower is a negative control, to which no reverse transcriptase was added in reverse transcription reactions. In the upper embryo, expression of the Xlens1 gene in the developing eyes (marked with an asterisk) has been detected.

FIG. 3 shows a result of detection of the BMP-10 gene performed in accordance with one example of the present invention. Of the two embryos, the upper is a detected example and the lower is a negative control, to which no reverse transcriptase was added in reverse transcription reactions. In the upper embryo, specific expression of the BMP-10 gene has been detected in the heart (indicatedbya dashed line arrow) and limb buds (indicated by a solid line arrow).

FIG. 4 is a schematic view showing the configuration of the automated whole-mount in situ RT-PCR apparatus system constructed in accordance with one example of the present invention.

FIG. 5 is a schematic view of heat blocks 1 and 2 for PCR included in the automated whole-mount in situ RT-PCR apparatus constructed in accordance with one example of the present invention.

FIG. 6 is an example of the user interface displaying the settings at the pretreatment stage in whole-mount in situ RT-PCR reactions in the monitor included in the automated whole-mount in situ RT-PCR apparatus system constructed in accordance with one example of the present invention.

### BEST MODE FOR CARRYING OUT THE INVENTION

The object, characteristics, and advantages of the present invention as well as the idea thereof will be apparent to those skilled in the art from the descriptions given herein. It is to be understood that the embodiments and specific examples of the invention described herein below are to be taken as preferred examples of the present invention. These descriptions are only for illustrative and explanatory purposes and are not intended to limit the invention to these embodiments or examples. It is further apparent to those skilled in the art that various changes and modifications may be made based on the descriptions given herein within the intent and scope of the present invention disclosed herein.

Whole-Mount in situ RT-PCR Method

In this embodiment, PCR is performed in situ to reverse transcribe mRNA and amplify the synthesized cDNA in whole-mount animal embryos or tissues (hereinafter called whole-mount in situ RT-PCR) . This embodiment consists of the following steps:

(1) Fixation of Animal Embryos or Tissues

To fix embryos or tissues, a buffer containing about 10% formalin or about 4% paraformaldehyde is used. Saline, a phosphoric acid buffer, etc. can be used as a buffer used for a solvent. Although these are fixatives most generally used, other fixatives, such as glutaraldehyde and DMSO/methanol, may be used. Alternatively, a mixed solution of these may be used as a fixative.

(2) Pretreatment of Fixed Samples

Since whole-mount embryos or tissues have a few dozens to a few hundreds of times the thickness as compared with thin sections, which are a few micrometers to a few dozens of micrometers, the permeability of probes, reagents, etc. becomes a big problem when gene expression is detected. If the permeability of probes is poor, the resulting signals will be weak and specific signals cannot be detected. Thus, the permeability of samples is improved by pretreating fixed samples.

The inventors found out that removal of lipid improves the permeability of samples and introduced a step of removing lipid as apretreatment of fixed samples. Specifically, in this embodiment, lipid is removed by xylene treatment.

(3) In Situ RT-PCR

The in situ hybridization method requires synthesis of labeled RNA or DNA from CDNA etc. by the in vitro transcription method, PCR, primer extension, nick translation, etc. in synthesizing probes. CDNA etc. of the gene to be analyzed was necessary as a template for such synthesis. In contrast, in situ RT-PCR, as long as the sequence of the gene to be analyzed is known, only synthesis of primers based thereon is required and cDNA of the gene is not. Now that the genome projects are in progress or have been completed in various animals, gene sequences can easily be obtained from gene banks and the present invention can be implemented in a simple and convenient manner. Further, in the present invention, since gene expression is detected in whole-mount animal embryos or tissues, there is no need to prepare thin-sliced sections; simple and convenient detection of gene expression is possible in this respect as well. Thus, according to the implementation of the present invention, gene expression can be investigated in a simple and convenient manner. This convenience enables comprehensive investigation of expression of numerous genes or DNA fragments, which have been still under study, by synthesizing primers based on information such as Expression Sequence Tag (EST).

One possible problem in performing RT-PCR in whole-mount is amplification of genomic DNA. When introduced primers hybridize to the genomic DNA, the gene is amplified there and detected as nonspecific signals, thereby inhibiting the detection of specific signals. Thus, in section-based in situ RT-PCR, amplification from the genomic DNA was suppressed by performing DNase treatment before reverse transcription reactions to degrade the genomic DNA. However, DNase treatment seriously damages tissues. Tissues treated with DNase cannot bear high temperature treatment in the subsequent PCR stage and their structures tend to be broken down.

Therefore, in the examples of the present invention, dNTP analogs that have the property of lowering the dissociation temperature of the nucleic acid duplex than the dissociation temperature of the nucleic acid duplex composed of usual dNTPs when incorporated into nucleic acid, were used. Accordingly, in the denaturation step of PCR, it is possible to specifically amplify cDNA by setting the denaturation temperature to a degree at which the genomic DNA does not dissociate, while cDNA dissociates. By this method, the genomic DNA is not amplified even without DNase treatment, because it is not denatured during PCR.

For example, a molecule that forms only single hydrogen bond in complementarily forming a duplex can be the dNTP analog in this case. In the examples of the present invention, a dNTP/analog mixture in mRNA Selective PCR Kit (TAKARA) was used. The denaturation temperature in PCR is typically set at 94° C. or higher, but when this dNTP/analog mixture is used, it may be set at 90° C. or lower, more preferably at 85° C. or lower. The lowest limit may be set at preferably 70° C. or higher, more preferably at 80° C. or higher.

It is preferable to vibrate a reaction mixture containing whole-mount animal embryos or tissues during a PCR reaction, which can enhance permeability of a reaction mixture to the embryos or tissues.

(4) Labeling of the Amplified DNA and Detection of the Label

To detect the amplified DNA, DNA is labeled and the label is used as a tag for detection of amplified DNA. Amplified DNA may be detected by, for example, incorporating labeled dNTP into DNA and detecting the label.

In the examples of the present invention, the amplified DNA is labeled by adding digoxigenin-labeled dUTP (hereinafter described as dig-dUTP) to the reaction in synthesizing cDNA and incorporating it into DNA to be synthesized. Finally, the amplified DNA is immunohistochemically detected using an antibody against digoxigenin.

Examples according to the present invention are hereinafter described in detail. Unless otherwise explained, methods described in standard sets of protocols such as J. Sambrook and E. F. Fritsch & T. Maniatis (Ed.), “Molecular Cloning, a Laboratory Manual (2nd edition), Cold Spring Harbor Press and Cold Spring Harbor, N.Y. (1989); and F. M. Ausubel, R.Brent, R. E. Kingston, D. D. Moore, J. G. Seidman, J. A. Smith, and K.Struhl (Ed.), “Current Protocols in Molecular Biology,” John Wiley & Sons Ltd., or alternatively, their modified/changed methods are used. When using commercial reagent kits and measuring apparatus, unless otherwise explained, protocols attached to them are used.

### EXAMPLE 1

In this example, expression of cardiac-specific troponin I is detected by whole-mount in situ RT-PCR.

(I) Fixation of Whole-Mount Embryos or Tissues

Xenopus laevis embryos at stage 25 are enveloped in the transparent vitellin membrane and further protected with a jelly coat. Jelly is removed by dissolving in a 2% cysteine solution and the vitellin membrane by stripping with forceps. The embryos from which the jelly coat and the vitellin membrane have been removed are fixed in MEM (0.1M MOPS [pH 7.4]−2 mM EGTA-1 mM MgSO₄) buffer containing 4% paraformaldehyde (hereinafter described as PFA fixative) at 4° C. for 12 hours.

In the case of X. laevis embryos, the method according to the present invention can be applied to embryos from egg stage to around stage 45, but embryos from stage 10 to 45 are more preferable.

Reactions after fixation are performed using 0.5 ml microcentrifuge tubes or multi-well plates (e.g., 96-well plastic plates). It is not necessary to change containers for each reaction. It is preferable to use tubes or plates compliant with the specifications for the thermal cycler so that they can be also used for amplifying cDNA in the thermal cycler.

Fixed embryos are dehydrated by sequentially treating for 5-10 min with the following solutions. Dehydrated embryos can be preserved at −20° C. for more than a few months.
(1) MEM buffer
(2) 25% EtOH in MEM buffer
(3) 50% EtOH
(4) 75% EtOH
(5) 100% EtOH (twice)
(II) Pretreatment
   (A) Rehydration

As pretreatment of whole-mount in situ RT-PCR, first, dehydrated embryos are rehydrated by sequentially treating with the following solutions, each for 5 min unless otherwise described.
(1) 75% EtOH
(2) 50% EtOH
(3) 25% EtOH in PTw

(PTw refers to 0.1% Tween 20 in PBS; hereinafter described as PTw)
(4) PTw (twice)
   (B) Acetylation

Next, to suppress non-specific binding of nucleic acids having anions to proteins, especially to basic proteins, in fixed embryos by neutralizing cations in the proteins, amine that is the cause of the basicity is acetylated. Here, amine is acetylated by treating fixed embryos with acetic anhydride.
(1) 0.1M triethanolamine
(2) 12.5 μl of acetic anhydride in 5 ml of 0.1M triethanolamine
(3) Another 12.5 μl of acetic anhydride in the solution used in (2), after the treatment (2)
   (C) Removal of lipid

Next, to improve permeability of primers, reagents, etc. into embryos or tissues, lipid contained therein is removed. Here, the lipid is removed by treating embryos with xylene. Specifically, embryos are sequentially treated with following solutions. The treatment time is 5 min unless otherwise described.
(1) PTw (twice)
(2) 25% EtOH in PTw
(3) 50% EtOH
(4) 75% EtOH
(5) 100% EtOH
(6) Xylene+100% EtOH (1:1)
(7) Xylene 2 hours
(8) Xylene+100% EtOH (1:1)
(9) 100% EtOH (three times) 3 min for each
(III) Reverse transcription reaction

In pretreated embryos or tissues, first, by performing a reverse transcription reactions using mRNA of the gene to be analyzed, cDNA of the gene is synthesized in situ.

Dehydrated embryos are dried for a short time but not completely dried, put in 38 μl of the following buffer, and incubated at room temperature for 60 min. Subsequently, the RT buffer is discarded, an equivalent amount of RT buffer is added, and incubated at 42° C. for 60 min. 5 U/μl AMV Reverse Transcriptase XL is then added and reverse transcription reaction is performed at 42° C. overnight. RT buffer:

| | |
|---|---|
| 2× mRNA Selective PCR Buffer I | 20 μl |
| 15 mM MgCl₂ | 10 μl |
| dNTP/analog mixture | 5 μl |
| 40 U/μl RNAse inhibitor | 1 μl |
| 100 μM down primer 1 | 2 μl |

Down primer 1 sequence:

| | |
|---|---|
| 5′-CTGATGGATGAAGTGCAATTAC-3′ | [SEQ ID NO: 1] |

(IV) Nucleic acid amplification reaction

After the reverse transcription reaction, the RT buffer is discarded and the following PCR buffer is added. PCR buffer:

| | |
|---|---|
| 2× mRNA Selective PCR Buffer I | 25 μl |
| 15 mM MgCl2 | 10 μl |
| dNTP/analog mixture | 5 μl |
| 125 mM dig-dUTP | 1 μl |
| 5 U/μl AMV optimized Taq | 5 μl |
| 100 μM upper primer | 2 μl |
| 100 μM down primer 2 | 2 μl |

Upper primer sequence:

| | |
|---|---|
| 5′-CAGCCTCTGCAACTGTCTG-3′ | [SEQ ID NO: 2] |

Down primer 2 sequence:

| | |
|---|---|
| 5′-GATCTACGGGCCAATCTC-3′ | [SEQ ID NO: 3] |

Using this reaction system, 30 cycles of PCR are performed under the following conditions to amplify cDNA.

| | | | |
|---|---|---|---|
| Conditions for 1 cycle: | Denaturation step | 84° C. | 1 min |
| | Annealing step | 45° C. | 2 min |
| | Extension step | 72° C. | 6 min |

(V) Label detection reaction

Finally, the region where troponin I is expressed is identified by detecting dig-dUTP incorporated into amplified nucleic acids using an anti-digoxigenin antibody (hereinafter described as anti-dig Ab).

First, the PCR buffer is discarded. Embryos are sequentially treated under the described conditions of period, temperature, and time using the following solutions. Each treatment is performed only one time at room temperature unless otherwise described.

| | | | | |
|---|---|---|---|---|
| (1) | 75% EtOH | 3 min | | |
| (2) | 2× SSC | 10 min | | |
| (3) | 1× SSC | 10 min | 3 times | 65° C. |

(4) MAB (100 mM maleic acid, 150 mM sodium chloride, pH 7.5) 5 min twice
(5) 2% Blocking Reagent (a trademark of Roche) and 20% lamb serum in MAB 60 min

(6) Alkaline phosphatase-conjugated anti-dig Ab (2,000-fold dilution with MBA) 4 hours

| | | |
|---|---|---|
| (7) | Wash with MAB | 10 min twice |
| (8) | Wash with MAB | 4° C. overnight |
| (9) | Wash with MAB | 60 min twice |
| (10) | Phosphatase buffer | 5 min twice |

Finally, an alkaline phosphatase substrate BM purple (a trademark of Roche) is added and color is developed at 37° C. During the color development, the embryos are observed under a stereoscopic microscope and, the reaction is stopped by adding PFA fixative when it is possible to distinguish between specific and non-specific signals

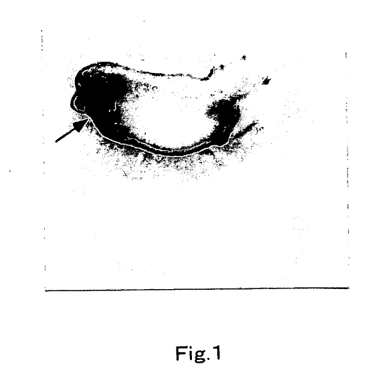

As shown in FIG. 1 (the shaded region indicates specific signals), troponin I expression has been detected specifically in the heart.

### EXAMPLE 2

In this example, expression of the Xlens1 gene that is specifically expressed in the presumptive lens epithelium is detected by whole-mount in situ RT-PCR.

Methods are identical to those used in Example 1 except the primers used for the reverse transcription reaction and nucleic acid amplification reaction and thus the details of the methods are omitted here. For primers, oligonucleotides with the following sequences were used:

Down primer1 sequence:

| | |
|---|---|
| 5′-GGCTGTGGTACATCAGTACT-3′ | [SEQ ID NO: 4] |

Upper primer sequence:

| | |
|---|---|
| 5′-CCTCTGGAGGCAGGAGAAG-3′ | [SEQ ID NO: 5] |

Down primer2 sequence:

| | |
|---|---|
| 5′-GCTCTGGATATAACCCTCAGA-3′ | [SEQ ID NO: 6] |

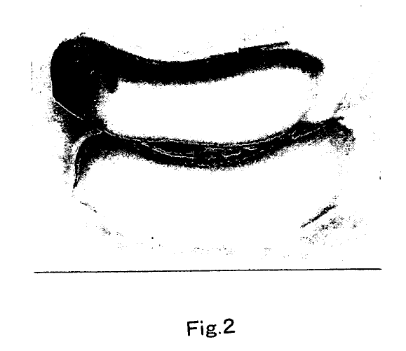

As a result, as shown in FIG. 2 (the shaded region indicates specific signal), expression of the Xlens1 gene was specifically detected in the eyes.

### EXAMPLE 3

In this example, expression of the mouse BMP-10 gene that is expressed in the limb buds and heart, using 9 to 13-day mouse embryos, instead of X. laevis embryos used in Examples 1 and 2, by whole-mount in situ RT-PCR.

(I) Fixation of Whole-Mount Embryos or Tissues

Day 10 mouse embryos were isolated from pregnant female mice and fixed at 4° C. overnight in PBS containing 4% paraformaldehyde +0.5% glutaraldehyde. Fixed embryos are dehydrated in the same manner as in Example 1 using PBS, instead of MEM, as the buffer.

(II) Pretreatment

The fixed mouse embryos are rehydrated in the same manner as in Example 1 (II) (A) and then incubated in 10 μg/ml of proteinase K solution for 5 min at room temperature, followed by acetylation as in Example 1 (II) (B) and then by lipid removal as in Example 1 (II) (C).

In this pretreatment step, it is preferred to optimize the concentration of proteinase K solution and proteinase K treatment time in advance, for each of the proteinase K lots and sizes of mouse embryos and tissues.

(III) Reverse Transcription Reaction

In pretreated mouse embryos, cDNA of the BMP-10 gene is synthesized in situ by performing reverse transcription reaction in a same manner as in Example 1 using the following primer.

| | |
|---|---|
| Primer sequence: | |
| 5′-TCCGTAGATCTCTGTTGATAC-3′ | [SEQ ID NO: 7] |

(IV) Nucleic Acid Amplification Reaction

Next, PCR is performed in the same manner as in Example 1 using the following primers.

| | |
|---|---|
| Primer sequence: | |
| 5′-CACACTGCTGCAGAGCATG-3′ | [SEQ ID NO: 8] |
| Primer sequence: | |
| 5′-TGATACTAAGACCAGCATGCT-3′ | [SEQ ID NO: 9] |

(V) Label Detection Reaction

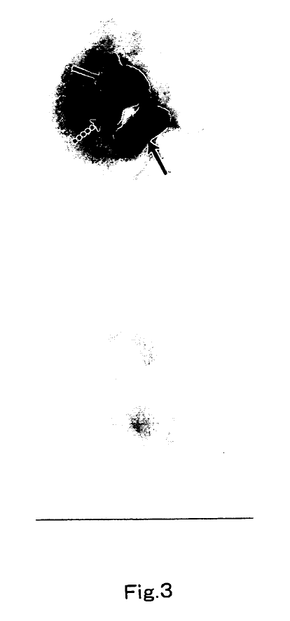

Finally, the region where the BMP-10 gene is expressed is identified by detecting dig-dUTP incorporated into amplified nucleic acids with an anti-digoxigenin antibody. The region is detected in the same manner as in Example 1 except that MAB is replaced with TBST. As a result, as shown in FIG. 3, expression of the BMP-10 gene was specifically detected in the heart and limb buds.

### Other Embodiments

In the above-described examples, X. laevis and mice are used as materials, the methods are also applicable to vertebrates such as zebra fish and chick as well as invertebrates such as Drosophila. This is because, in the case of in situ hybridization or whole-mount in situ hybridization, almost the same methods can be also used for such animal species. However, the amount of non-specific signal may vary according to genes and tissues to be used, in which case also non-specific signal can be reduced by modifying not the treatment step of nucleic acids such as probe hybridization but the pretreatment of tissues carried out before that. The present invention concerns general methods for reducing non-specific signal in the treatment step of nucleic acids that have been subjected to pretreatment (i.e., reverse transcription reactions, amplification reactions, and detection reactions of nucleic acids) as well as that of pretreatment. The present invention is therefore applicable to any animal species.

Although proteinase K was used for protease treatment in the above examples, other kinds of protease, papain, etc. may be used.

Although only the target mRNA was reverse transcribed using primers specific to the mRNA in the reverse transcription reaction in the above examples, total mRNA may be reverse transcribed using random primers or oligo dT as primer. Even in such a case, since primers specific to the target cDNA in PCR are used, only the cDNA is specifically amplified.

Although PCR was used as amplification reaction of nucleic acid in the above examples, for example, RNA polymerase-based nucleic acid amplifying methods or other nucleic acid amplifying methods such as the chain substitution amplifying methods like ICAN (a registered trademark of TAKARA) may be used.

Although amplification of genomic DNA was prevented by using dNTP analogs for synthesis and amplification of cDNA and taking advantage of differences in denaturation temperature between cDNA duplex and genomic DNA duplex in the above examples, cDNA may be amplified by degrading genomic DNA with DNase in advance and subsequently reverse transcribing mRNA. However, partial degradation of genomic DNA can result in formation of short oligonucleotides, thereby decreasing denaturation temperatures of genomic DNA. It is therefore preferable either to degrade genomic DNA almost completely to the extent that it cannot be amplified by PCR or not to perform DNase treatment. Among these, it is most preferable not to perform DNase treatment.

Although DNA to be synthesized was labeled with dig-dUTP incorporated during amplification of cDNA in the examples of the present invention, DNA may be labeled by using labeled primers that have been labeled in advance with digoxigenin etc.

In addition, although digoxigenin was used as a DNA labeling molecule, a label for amplified DNA may be the one detectable by spectroscopic, optical, biochemical, immunological, enzyme chemical, radiochemical or other means. Examples of such a label include, besides digoxigenin, enzymes such as peroxidase and alkaline phosphatase, radioactive labels such as ³²P, isotope, biotin, fluorochrome, fluorescent substance, coloring substance, etc.

Automated Whole-Mount in situ RT-PCR Apparatus

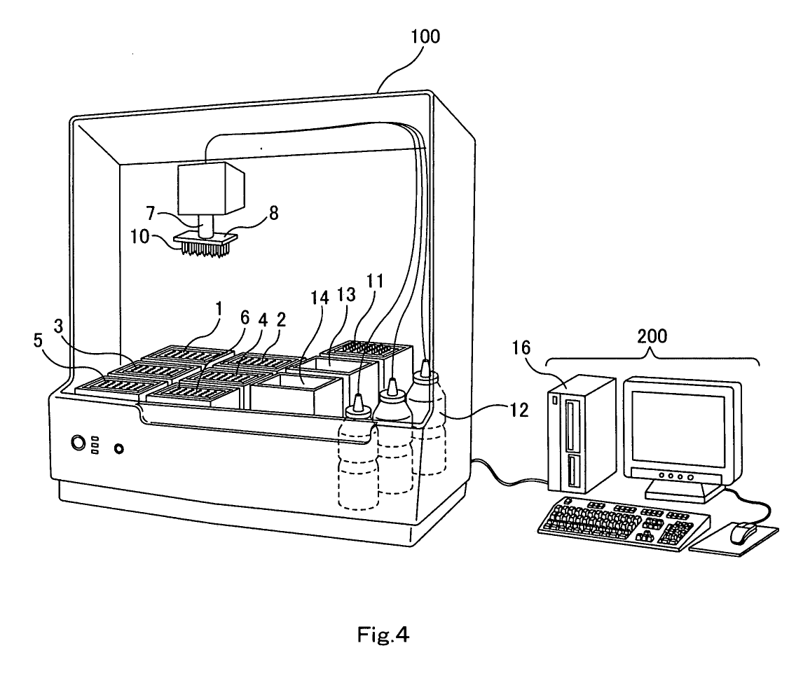

The whole-mount in situ RT-PCR method described above can be performed manually, as can the conventional experimental techniques. However, when treating a large number of samples, automation of processes can increase efficiency. For example, the apparatus as shown in FIG. 4 is conceivable. Since operations performed by each part inside the apparatus, such as moving a container containing samples, discarding a solution in a container, injecting fluid into a container, and vibrating a container, have been realized in apparatus such as automatic genomic DNA extractors and automated PCR systems, etc. Those skilled in the art can thus design such individual structures in the apparatus; description of the mechanic details of such structures is omitted here.

In the automated whole-mount in situ RT-PCR apparatus 100 in this embodiment, as shown in FIG.4, when a PCR plate with embryos has been placed in a PCR heat block 1 or 2 inside the apparatus, reactions are automatically performed up to the final step of coloring reaction according to a program. In the heat blocks 1 and 2, different reaction conditions such as temperatures and periods can be set for each column and each row. The PCR plate used here has a form of connected microtubes, with a high thermal efficiency and a thin tube wall.

Blocks 3 and 4 and blocks 5 and 6 adjacent to the heat blocks 1 and 2 are the places where primers used for RT reactions and PCR, performed in the heat block 1 and the heat block 2, respectively, are prepared. The plates for this purpose are the same as those for the reaction.

The apparatus includes an arm 7 for transferring fluid into and from the wells of the PCR plates. A tip 8 of the arm 7 is provided with nozzles at the place corresponding to the wells on the PCR plates. The tip 8 is exchangeable: for example, when a 24-well plate or a 96-well plate is used, the tip 8 with 24 or 96 nozzles can be selectively attached. Pipetman tips 10, which are coated with elastic materials such as rubber and silicone so as not to damage samples, are attachable to the tip of the nozzles and are automatically detachable therefrom. In a tip place 1 inside the apparatus, tip stands in which tips 11 are set in holes corresponding to the location of the nozzles of the tip 8 selected are placed. The tip stands with tips 11 are stackable. After the tips 11 are used and the empty stand is removed, another set of tips 11 set in the next stand become available.

The arm 7 is movable lengthwise and breadthwise and can be placed right above the heat blocks 1 and 2. In addition, the arm 7 can expand and contract vertically and can lower its tip up to right above the fixed position of the plate. The arm 7 therefore makes it possible to inject fluid into the wells of the plate placed in the fixed position of the heat blocks 1 and 2 or to remove fluid from the wells.

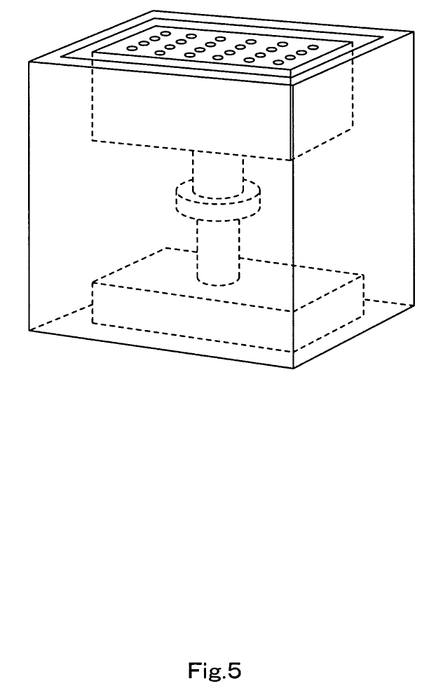

As shown in FIG. 5, these heat blocks 1 and 2 can vibrate reactants during PCR reactions by making 3D movements in the same manner as a belly dancer shaker does. It is also possible to vibrate reactants during PCR reactions by providing each heat block with an ultrasonic wave generator. In this case, vibration conditions, such as the frequency and strength of ultrasonic waves as well as period to apply ultrasonic waves, can be set for each column and each row of the heat blocks 1 and 2.

Reagents are incorporated into the inside of the apparatus through the tube inserted into reagent bottles 12 for supplying the reagents used for the reactions and released from the tip of the nozzles of the arm tip 8. Examples of reagents include distilled water, PBS, EtOH, RT buffer, and PCR buffer, prepared in the reagent bottles 12 required for reactions. Box 13 and box 14 placed inside the apparatus are for the disposal of tips and reagents, respectively.

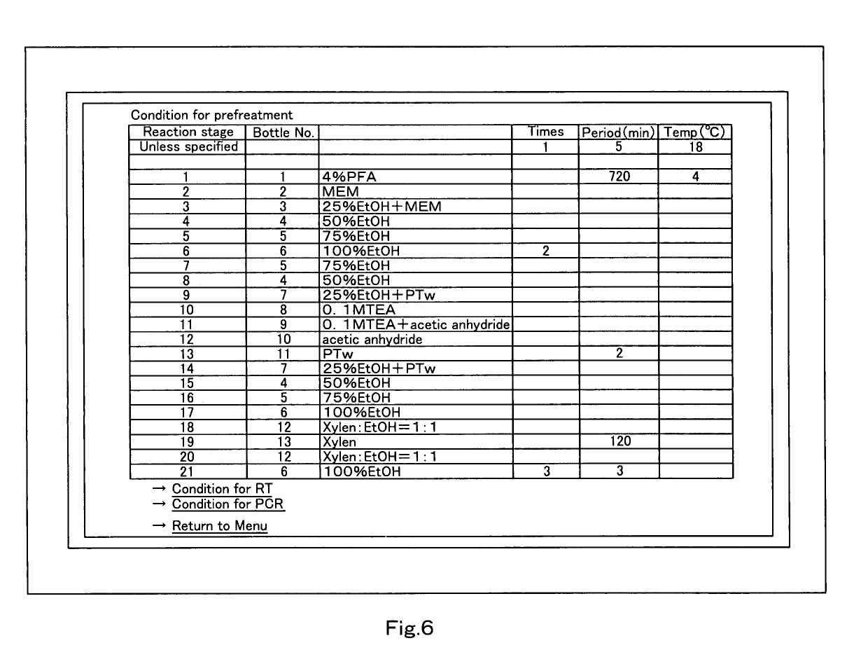

The automated whole-mount in situ RT-PCR apparatus 100 constitutes an automated whole-mount in situ RT-PCR apparatus system together with a computer system 200 and all operations of the apparatus are controllable using a computer 16 included in the system 200. Operations of the apparatus are, for example, operation of the arm 7, temperature control (including PCR conditions) of the heat blocks 1 and 2, vibration of the heat blocks 1 and 2 etc. The computer control of the system enables an experimenter to input the parameters for the pretreatment of X. laevis embryos in the user interface, for example as shown in FIG. 6.

Using the automated whole-mount in situ RT-PCR apparatus system with the above-described configuration, whole-mount in situ RT-PCR is performed on X. laevis embryos at stage 25 in accordance with Example 1. First, X. laevis embryos from which jelly and vitelline membrane have been removed are put in a 24-well plastic plate, which is placed in the heat block 1 set at 4° C. in advance. The arm 7 moves over to the plastic plate to inject and remove solutions required for the individual steps of fixation, dehydration, rehydration, acetylation, and lipid removal. The heat block 1 is controlled so as to have suitable temperature in each step.

Subsequently, the RT buffer containing AMV Reverse Transcriptase XL but not primers is added, once removed, and the same buffer is added again. Then, primers for each sample are added from the plate on the block 3 to carry out reverse transcription reaction. After the reaction, the RT buffer is discarded, the PCR buffer without primers is added, and then primers for each sample is added from the block 4 to carry out PCR.

Finally, after antigen-antibody reactions were performed using the alkaline phosphatase-conjugated anti-dig Ab, reactions stop immediately before coloring reactions and a buzzer sounds.

The experimenter takes out the plate from the automated whole-mount in situ RT-PCR apparatus 100, adds BM purple (a trademark of Roche), the substrate of alkaline phosphatase, and develops color at 37° C. Embryos are observed with a stereoscopic microscope on the way. When the intense of signal has reached the point where it is possible to distinguish between specific signal and non-specific signal, reactions are stopped by adding PFA fixative.

For the final coloring reaction, it is possible to determine when to stop the reaction by installing a built-in CCD camera and a monitor in the whole-mount in situ RT-PCR automation apparatus 100, adding a substrate inside the apparatus 100, and observing the reaction on the monitor outside the apparatus 100. Further, the program may be designed such that reaction may stop when the contrast between the background and the signal has reached to or exceeded a certain level, or the average/total strength of signal has reached to or exceeded

1. A method for detecting gene expression in an animal embryo or tissue, comprising the steps of: in a whole-mount animal embryo or tissue,
synthesizing a cDNA from an RNA;
amplifying a nucleic acid using the cDNA synthesized by the step of synthesizing the cDNA as a template; and
detecting the nucleic acid amplified by the step of amplifying the nucleic acid.

2. The method for detecting gene expression of claim 1, wherein the animal is a vertebrate.

3. The method for detecting gene expression of claim 2, wherein the vertebrate is Xenopus laevis.

4. The method for detecting gene expression of claim 2, wherein the vertebrate is a mouse.

5. The method for detecting gene expression of claim 1, wherein the step of amplifying the nucleic acid comprises the steps of:
a denaturation step in which a nucleic acid duplex is denatured to a single strand;
an annealing step in which a primer hybridizes to the nucleic acid that has became the single strand in the denaturation step; and
an extension step in which a nucleic acid having a sequence complementary to the nucleic acid that has become the single strand is synthesized by using the nucleic acid that has become the single strand as a template and extending from the primer.

6. The method for detecting gene expression of claim 5, wherein the step of amplifying the nucleic acid is performed by polymerase chain reaction (PCR).

7. The method for detecting gene expression of claim 1, wherein the whole-mount embryo or tissue is vibrated while the nucleic acid is being amplified.

8. The method for detecting gene expression of claim 5, wherein the nucleic acid is amplified by specifically using the synthesized cDNA as a template in the extension step, without substantially denaturing genomic DNA in the denaturation step in the step of amplifying the nucleic acid.

9. The method for detecting gene expression of claim 5, wherein the denaturation step is performed at 90° C. or lower in the step of amplifying the nucleic acid.

10. The method for detecting gene expression of claim 9, wherein, the inclusion of dNTP analogue in a reaction in the step of amplifying a nucleic acid and the inclusion of the dNTP analogue in the duplex consisting of the cDNA and its complementary strand generated in the extension step cause the dissociation temperature of the duplex consisting of the cDNA and its complementary strand to be 90° C. or lower.

11. The method for detecting gene expression of claim 10, wherein the dNTP analog is derived from a dNTP/analog mixture in mRNA Selective PCR Kit (a trademark of TAKARA).

12. The method for detecting gene expression of claim 1, further comprising the step of treating the whole-mount embryo or tissue with a protease.

13. The method for detecting gene expression of claim 1, further comprising the step of acetylating an amine in the whole-mount embryo or tissue.

14. The method for detecting gene expression of claim 13, wherein the step of acetylating the amine comprises treating the whole-mount embryo or tissue with acetic anhydride.

15. The method for detecting gene expression of claim 1, further comprising the step of removing a lipid in the whole-mount embryo or tissue.

16. The method for detecting gene expression of claim 15, wherein the step of removing the lipid comprises treating the whole-mount embryo or tissue with xylene.

17. The method for detecting gene expression of claim 1, wherein the method for detecting gene expression is performed in a microcentrifuge tube or by using a multiwell plate.

18. A kit for detecting gene expression in a vertebrate embryo or tissue comprising:
(a) a reverse transcriptase that synthesizes a cDNA from an RNA;
(b) a nucleic acid polymerase that polymerizes a nucleic acid;
(c) a dNTP analog that lowers the dissociation temperature of the nucleic acid than the dissociation temperature at which dNTP is used, when the dNTP analog is present in a double-stranded nucleic acid; and
(d) a labeled dNTP.

19. The kit of claim 18, further comprising:
(a) an antibody that recognizes a label in the labeled dNTP and that is bound to an enzyme for detecting a localization of the label; and
(b) a substrate for the enzyme for detecting the localization.

20. The kit of claim 18, further comprising a fixed embryo or tissue.

21. The kit of claim 18, comprising a dNTP/analog mixture in mRNA Selective PCR Kit (a trademark of TAKARA) as the dNTP analog.

22. The kit of claim 18, wherein the labeled dNTP is a dNTP labeled by digoxigenin (a trademark of Roche).

23. An apparatus for performing the method for detecting gene expression of claim 1.

24. An apparatus for performing the method for detecting gene expression of claim 23, the apparatus comprising a device for vibrating the whole-mount embryo or tissue while the nucleic acid is being amplified.
